(19) United States
(12) Patent Application Publication
(10) Pub. No.: US 2014/0066496 A1
(43) Pub. Date: Mar. 6, 2014

(54) Composition and Methods for Anti-Macrofouling Treatment of Polymers

(71) Applicants: Nikhil A. Gunari, Etobicoke (CA); Mallika Das, Etobicoke (CA); Gilbert C. Walker, Mississauga (CA)

(72) Inventors: Nikhil A. Gunari, Etobicoke (CA); Mallika Das, Etobicoke (CA); Gilbert C. Walker, Mississauga (CA)

(73) Assignee: THE GOVERNING COUNCIL OF THE UNIVERSITY OF TORONTO, Toronto, ON (CA)

(21) Appl. No.: 14/110,992

(22) PCT Filed: Apr. 11, 2012
(86) PCT No.: PCT/CA2012/000347

§ 371 Date: Nov. 21, 2013

Related U.S. Application Data
(60) Provisional application No. 61/474,114, filed on Apr. 11, 2011.

(51) Int. Cl. A61K 31/352

(52) U.S. Cl. CPC A61K 31/352
USPC 514/453

(57) ABSTRACT

There is herein provided a composition comprising azadirachtin, preferably neem oil, and methods of use thereof for inhibiting macrofouling on a polymer in an aquatic environment.

### FIELD OF THE INVENTION

The invention relates generally to polymers comprising a naturally occurring anti-macrofouling agent and methods of preparing the same. The treated polymers are particularly useful in aquaculture applications.

### BACKGROUND OF THE INVENTION

Natural and synthetic polymers, such as jute, hemp, flax, nylon, polyesters, polypropylene, and polyethylene have a wide variety of commercial applications. A primary commercial application of such natural and synthetic polymers is the production of fibers and ropes that can be used as is or to construct, for example, nets for the fishing and fish farming industries. Nylon, i.e., the monofilament fiber thereof, can be woven, twisted or knotted to form a twine which is subsequently further woven to form the desired mesh size of the net. Nylon is widely used in the fish farming industry due to its excellent properties and low production cost. Cage aquaculture and set-net fishery are methods widely used in fish farming industry, however, both typically suffer biofouling, which can directly affect fish health, reduce cage life, and increase the service costs. Severe invasion of nets by biofouling species reduces dissolved oxygen in the nets and, hence, impedes fish growth.

Since the emergence of fish farming, nets, such as nylon nets, have been traditionally treated with heavy metal-based paints to prevent biofouling. The bioaccumulation and biomagnification of heavy metals are an environmental hazard. Moreover, most antifouling coatings applied to nets readily wash out in the water, diminishing the antifouling efficacy, and require repeated mechanical cleaning of nets to eliminate the accumulated biofouling species. Such repeated cleanings of the nets lead to the loss of fiber strength and, hence, early breakdown.

The major drawback of traditional biocides, which comprise heavy metals, is the environmental toxicity and the short-time efficacy. There remains a need to identify environmentally friendly, metal-free antifouling products capable of long-term antifouling effects.

### SUMMARY OF THE INVENTION

The present invention provides a natural product-based composition for use in inhibiting the growth of undesirable macrofouling species on a polymer surface in an aquatic environment. Methods of using the natural product-based composition to inhibit macrofouling are also provided.

In an aspect, there is provided a composition comprising azadirachtin, preferably neem oil.

In another aspect, there is provided a polymer soaked with the composition described herein.

In another aspect, there is provided a method of inhibiting macrofouling on at least one polymer in an aquatic environment comprising applying the composition described herein to the polymer.

In a further aspect, there is provided the composition described herein for inhibiting macrofouling of a polymer in an aquatic environment.

In a further aspect, there is provided use of the composition described herein for inhibiting macrofouling of a polymer in an aquatic environment.

### BRIEF DESCRIPTION OF THE DRAWINGS

Preferred embodiments of the invention will now be described, by way of example only, with reference to the drawings, in which:

FIG. 1 illustrates the effects of A) no treatment and B) treatment with neem seed oil on one square foot nylon nets following 5 weeks in water. Neem oil treated nets showed significantly limited macrofouling after 5 weeks.

FIG. 2 illustrates the effects of A) no treatment and B) treatment with neem seed oil on one square foot nylon nets following 11 weeks in water. Greatly reduced macrofouling was observed on neem oil treated nets.

FIG. 3 illustrates the effects of A) no treatment and B) treatment with neem seed oil on one square foot nylon nets following 20 weeks in water at a depth of 18 feet. Neem oil treated nets showed greatly reduced macrofouling compared to untreated nets after 20 weeks.

FIG. 4 illustrates the effects of A) no treatment and B) combination treatment with neem seed oil and linseed oil on one square foot nylon nets following 20 weeks in water at a depth of 18 feet. Combination neem oil/linseed oil-treated nets showed reduced macrofouling compared to untreated nets after 20 weeks.

FIG. 5 illustrates the effects of A) no treatment and B) combination treatment with neem seed oil, linseed oil and limonene on one square foot nylon nets following 20 weeks in water at a depth of 18 feet. Combination neem oil/linseed oil/limonene-treated nets showed reduced macrofouling compared to untreated nets after 20 weeks.

FIG. 6 illustrates the effects of A) triblock copolymer micellar solution (polycaprolactone-b/ock-poly(ethylene oxide)-block polycaprolactone triblock copolymer) (control) and B) 1% azadirachtin-loaded aqueous triblock copolymer micellar solution (polycaprolactone-b/ock-poly(ethylene oxide)-block polycaprolactone triblock copolymer) on one square foot nylon nets following 20 weeks in water at a depth of 18 feet. Aqueous block copolymer-encapsulated azadirachtin showed antifouling effects after 20 weeks compared to control alone, however, the anti-macrofouling effects were not as significant as observed in nets treated with neem oil.

FIG. 7 illustrates the effects of combination neem oil (10000 ppm azadirachtin)/linseed oil/limonene treatment, having a final azadirachtin concentration of ˜1000 ppm in the formulation, on a one square foot nylon net following 5 weeks in water at a depth of 18 feet.

### DETAILED DESCRIPTION OF INVENTION

Generally speaking, the embodiments described herein are directed to compositions for, and methods, of protecting a polymer surface, in particular to inhibit macrofouling on a polymer surface.

In an aspect, there is described herein a composition comprising, a high flashpoint biodegradable vegetable oil that absorbs into the fibers creating a hydrophobic environment at the interface of the fiber surface and bulk water. The fiber acts as a reservoir for the biocide that can diffuse to the interface to give targeted antifouling properties. The biocide being hydrophobic and biodegradable provides a slow release mechanism; a long lasting and environmentally friendly alternative to the currently used toxic heavy metal based paints.

As required, embodiments of the present invention are disclosed herein. However, the disclosed embodiments are merely exemplary, and it should be understood that the invention may be embodied in many various and alternative forms.

In an aspect, there is provided a composition comprising azadirachtin, preferably neem oil.

In a preferable embodiment, the composition further comprises at least one polymerized oil, preferably selected from the group comprising a linseed oil, a perilla oil, a poppy seed oil, a soybean oil, a walnut oil, a tung oil, and mixtures thereof.

In some embodiments, the composition comprises from 1 to 100% (v/v) neem oil.

In some embodiments, the composition comprises azadirachtin in a concentration from at least 1 ppm to 65,000 ppm.

In some embodiments, the composition comprises from >0 to 99% (v/v) of the at least one polymerized oil.

In some embodiments, the at least one polymerized oil is a boiled oil having an iodine number greater than 120.

In a preferable embodiment, the composition further comprises at least one essential oil derived from a fruit or a flower, preferably selected from the group comprising limonene, lavender, rose and mixtures thereof.

In an embodiment, the composition comprises linseed oil and limonene.

In some embodiments, block-copolymers may also be used in connection with the claimed compositions. Such block-copolymers should be capable of forming a micelle to encapsulate the active ingredient. Examples of such block-copolymers are disclosed in WO2010/045728 and WO2011/130857 and can be diblock, triblock or multiblock copolymers. Preferably, the block copolymer is biodegradable and is further preferably polycaprolactone-block-poly(ethylene oxide)-block polycaprolactone triblock copolymer.

In some embodiments, the composition further comprises a foul release agent. A foul release agent is an agent that enhances the release of a foulant, often by making a soft and/or slippery surface and/or exhibiting low adhesion to foulants. Exemplary foul release agents have properties that mimic the foul release properties of the fatty acids in, for example, an oil-based composition such as neem oil. Non-limiting examples of foul release agents include polydimethylsiloxane, chitosan, polyvinylpyrrolidone. Preferably, the foul release agent is polyvinylpyrrolidone.

In another aspect, there is provided a polymer soaked with the composition described herein. Preferably, the polymer is nylon in the form of a net.

In another aspect, there is provided a method of inhibiting macrofouling on at least one polymer in an aquatic environment comprising applying the composition described herein to the polymer.

In a preferable embodiment, the at least one polymer is selected from the group consisting of a synthetic or a natural polymer, preferably of nylon, high density polyethylene (HDPE), polyester, polyurethane, Teflon, cellulose and polypropylene.

In some embodiments, the at least one polymer is contained in at least one of a fiber, a rope, a sheet, a film, and a net, preferably a net.

In some embodiments, the composition inhibits macrofouling of at least one aquatic plant or aquatic animal selected from a group comprising an hydroid, a mussel, a tunicate, a barnacle, a bryozoan, an annelid, and a macroalgae.

In some embodiments, the applying of the composition to the at least one polymer comprises at least one of dipping, spraying, brushing, rolling and pouring the composition over the at least one polymer. In one embodiment, the composition is applied to the at least one polymer during the cold drawing stage of producing a polymer fiber, preferably the cold drawing process is executed on a draw twister machine.

In a further aspect, there is provided the composition described herein for inhibiting macrofouling of a polymer in an aquatic environment.

In a further aspect, there is provided use of the composition described herein for inhibiting macrofouling of a polymer in an aquatic environment.

As used herein, “macrofouling” refers to the growth of undesirable aquatic plants or animals on surfaces that are submerged in water. Non-limiting examples of organisms contributing to macrofouling include aquatic plants and animals such as hydroids, mussels, tunicates, barnacles, bryozoans, annelids, and macroalgae.

As used herein, “polymer” refers to a large molecule (macromolecule) composed of repeating structural units. The repeating units are typically connected by covalent bonds. A polymer can be natural or synthetic. Non-limiting examples of polymers are nylon, high density polyethylene (HDPE), polyester and polypropylene.

As used herein, “aquatic environment” refers to waters, including wetlands, which serve as a habitat for interrelated, and interacting communities and populations of plants and animals. Aquatic environment can be natural or man-made.

As used herein, “azadirachtin” refers to a chemical compound belonging to the limonoids. It is a secondary metabolite present in the neem tree seeds.

As used herein, “polymerized oil” refers to a cross-linked viscous oil. Non-limiting examples of a polymerized oil are a linseed oil, a perilla oil, a poppy seed oil, a soybean oil, a walnut oil, a tung oil, and mixtures thereof.

As used herein, the term “inhibit” refers to at least partial decrease or inhibition of macrofouling by the claimed compositions as compared to without.

As used herein, “iodine number” refers to the mass of iodine in grams that is consumed by 100 grams of a chemical substance. Examples of high iodine number (>120) oils are linseed, perilla, poppy seed, soybean, walnut, and tung oils.

As used herein, “cold drawing” refers to process of stretching polymer fibers to align polymer chains. It is typically performed after the material has been spun into filaments; by extruding the polymer melt.

As used herein, “draw twister” refers to a machine used to draw and twist large quantities of polymer fibers.

The following are examples that illustrate a method for the preparation of compositions of the present invention to fabricate protective films on a polymer surface. These examples are intended to illustrate the nature of such preparations and are not intended to be limiting in the scope of applicable composition and methods.

### EXAMPLES

### Example 1

Neem oil extraction and net treatment:

In a ball mill, 500 g of neem seeds were pulverized thoroughly at room temperature for 10 h. Pulverized neem seed powder was placed in a beaker. One L of water was added and mixed thoroughly. A watch glass was placed on the top of the beaker. The mixture was heated at 80° Celsius. The steam condensed on the watch glass and was allowed to drip back into the mixture. The oil floated on top. One square foot nylon net was dipped into the extracted oil. The net was then allowed to drip dry.

### Field Testing:

Field tests were carried out in a low energy test site in the ocean bay. Untreated (no coating) and treated (neem oil) one square foot nylon nets were immersed at a depth of 18 feet. Net test samples were photographed after: 1) 5 weeks, and 2) 11 weeks.

### Results:

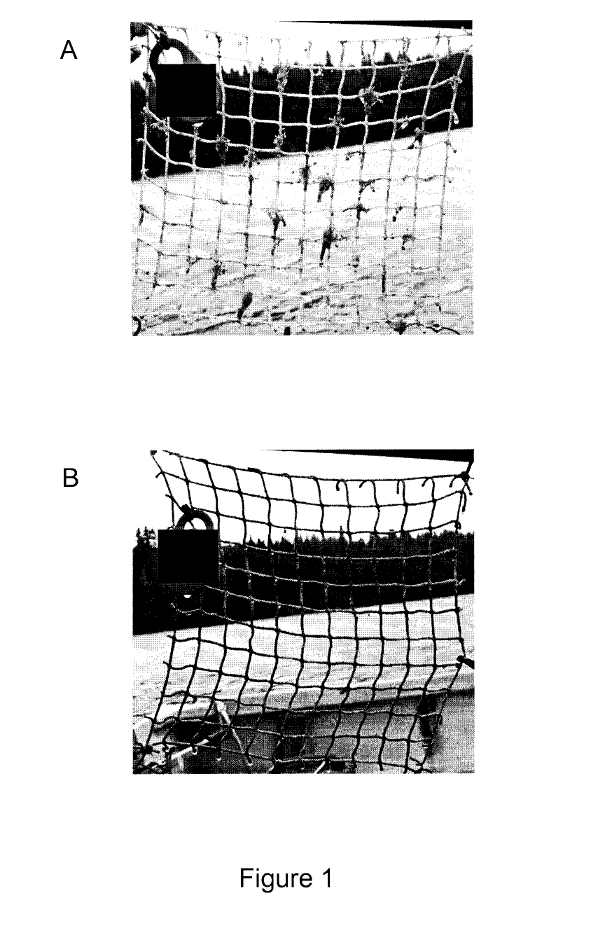

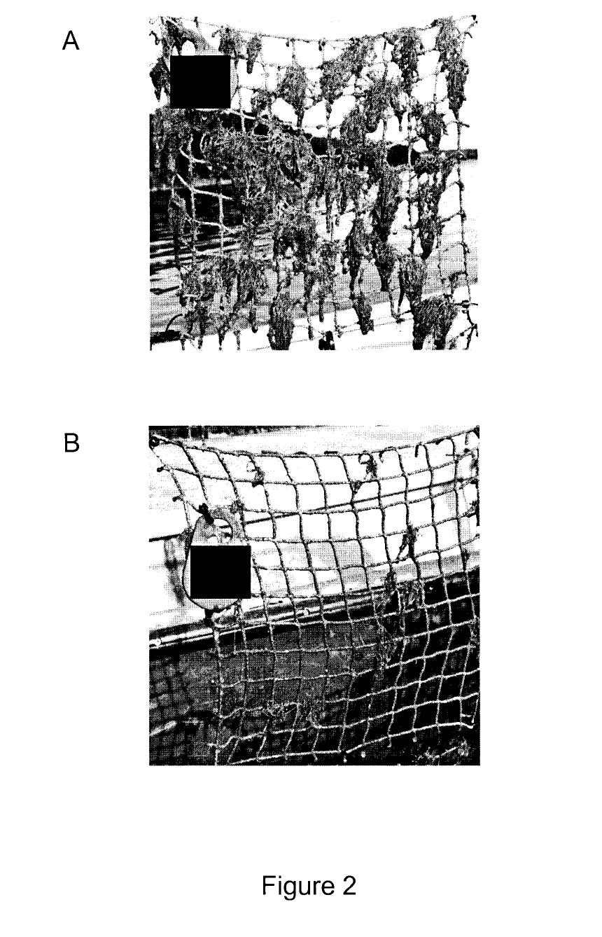

After 5 weeks in water, the untreated (no coating) net showed macrofouling (FIG. 1A), predominantly hydroids, whereas the neem oil treated net showed little to no macrofouling (FIG. 1B). After 11 weeks in water, the untreated (no coating) net was almost completely covered with hydroids (FIG. 2A), whereas macrofouling on the neem oil treated net was significantly reduced in comparison to untreated (FIG. 2B).

Neem oil treated nets showed very good long-term efficacy against macrofouling species.

### Example 2

### Net Treatment:

A one square foot nylon net was treated with neem oil (100 ml, ˜1000 ppm azadirachtin) by dip-treatment. The net was allowed to soak for 5 minutes. The net was then removed and allowed to drip dry in air. Twenty-two grams of nylon net soaked up approximately 8 grams of neem oil.

### Field Testing:

Field tests were carried out in a low energy test site in the ocean bay. Untreated (no coating) and treated (neem oil) one square foot nylon nets were immersed at a depth of 18 feet. Net test samples were photographed after 20 weeks.

Field Test Results:

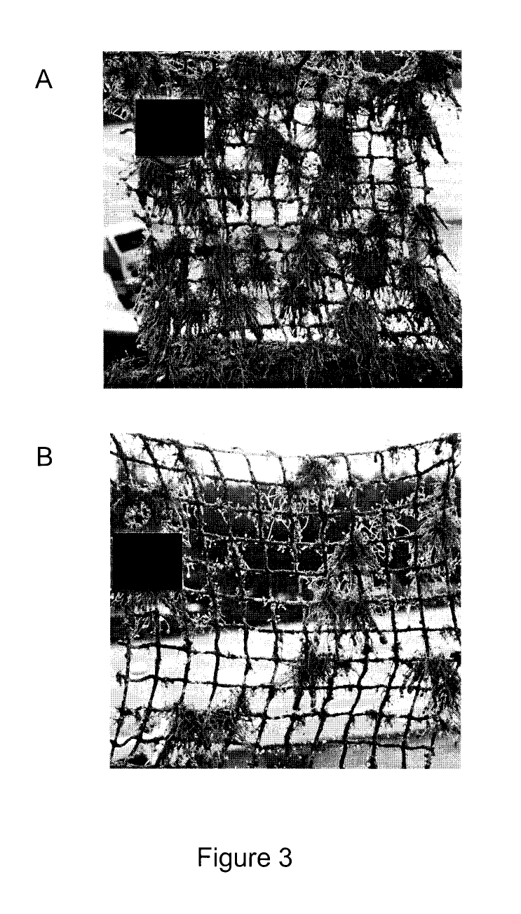

After 20 weeks in water, the untreated (no coating) net was almost completely covered with hydroids (FIG. 3A), whereas macrofouling on the neem oil treated net was significantly reduced in comparison to untreated (FIG. 3B).

Neem oil treated nets showed very good long-term efficacy against macrofouling species.

### Example 3

### Net Treatment:

Sixty mL of neem oil was mixed with 40 mL of polymerized linseed oil. The mixture was stirred for a half hour to allow thorough mixing. A one square foot nylon net was treated with the mixed oil composition. The net was allowed to soak for 5 minutes. The net was then removed and allowed to dry in air. Twenty-two grams of nylon net soaked up approximately 9 grams of neem oil.

### Results:

The net treated with the mixed oil composition dried in 2 days and was observed to be more dry, i.e. less oily, on its surface, than the neem only net, suggesting improved absorption and generally improving the cosmetic properties of the net. Air oxidation and polymerization of the linseed oil likely also contributed to a more dry net.

### Field Testing:

Field tests were carried out in a low energy test site in the ocean bay. Untreated (no coating) and treated (combination neem oil and linseed oil) one square foot nylon nets were immersed at a depth of 18 feet. Net test samples were photographed after 20 weeks.

### Field Test Results:

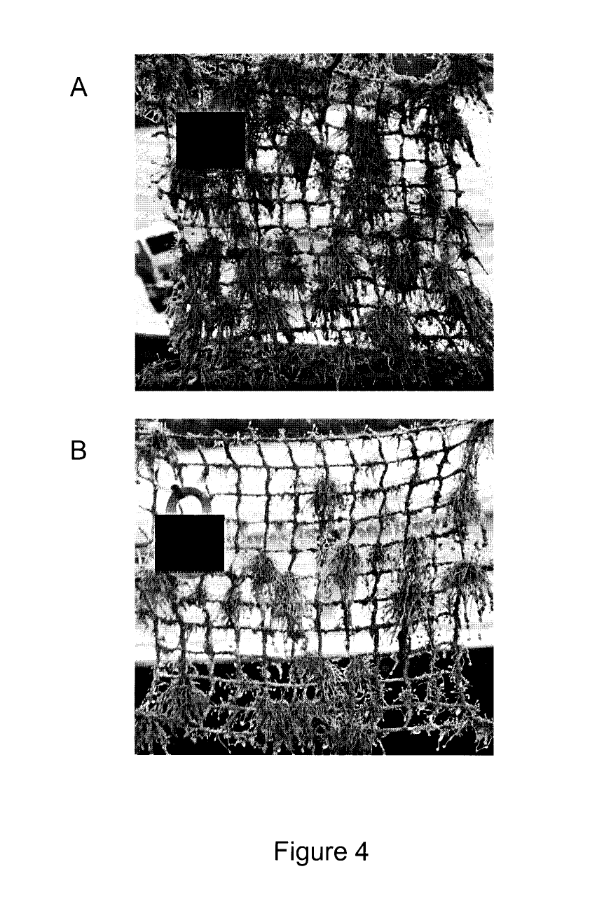

After 20 weeks in water, the untreated (no coating) net was almost completely covered with hydroids (FIG. 4A), whereas macrofouling on the combination neem oil and linseed oil treated net was reduced in comparison to untreated (FIG. 4B).

Combination neem oil and linseed oil treated nets showed very good long-term efficacy against macrofouling species.

### Example 4

### Net Treatment:

Fifty mL of neem oil was mixed with 40 mL of polymerized linseed oil and 10 mL of limonene. The mixture was stirred for half hour to allow thorough mixing. A one square foot nylon net was treated with the mixed oil composition. The net was allowed to soak for 5 minutes. The net was then removed and allowed to dry in air. Twenty-two grams of nylon net soaked up approximately 9 grams of neem oil.

### Field Testing:

Field tests were carried out in a low energy test site in the ocean bay. Untreated (no coating) and combination neem oil/linseed oil/limonene treated one square foot nylon nets were immersed at a depth of 18 feet. Net test samples were photographed after 20 weeks.

### Field Test Results:

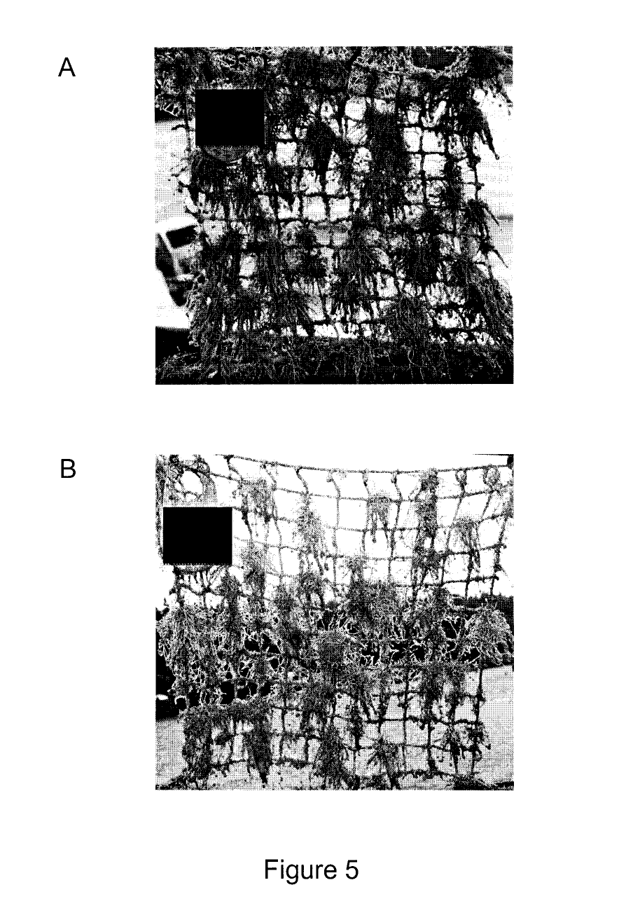

After 20 weeks in water, the untreated (no coating) net was almost completely covered with hydroids (FIG. 5A), whereas macrofouling on the combination neem oil/linseed oil/limonene treated net was reduced in comparison to untreated (FIG. 5B).

Combination neem oil/linseed oil/limonene treated nets showed very good long-term efficacy against macrofouling species.

### Example 5

### Formulation: Azadirachtin Nano-Encapsulated in Biodegradable Block Copolymer Micelles

### Materials and Methods

Polycaprolactone-block-poly(ethylene oxide)-block polycaprolactone triblock copolymer (Polymer Source) was used without further purification. Polycaprolactone-block-poly(ethylene oxide)-block polycaprolactone (polydispersity index 1.25, with number average molecular weight polycaprolactone-block-poly(ethylene oxide)-block polycaprolactone triblock copolymer (4,000-10,000-4,000) g/mol) was dissolved in chloroform to give a 2 wt %. Polystyrene-block-poly(ethylene oxide) (polydispersity index 1.05, number average molecular weight for polystyrene 3,600 g/mol and for poly(ethylene oxide) 67,000 g/mol) was dissolved in Milli Q water. Polystyrene-block-poly(ethylene oxide) was used as a phase transfer agent. The triblock copolymer solution was introduced into a separatory funnel and the aqueous solution containing the phase transfer agent was added to the funnel. The two solutions were separated after two hours. The aqueous solution was turbid.

The aqueous triblock copolymer micellar solution was then loaded with 0.2-g/l azadirachtin (1%). The composition was mixed vigorously for 30 minutes and then allowed to stand.

### Net Treatment:

One square foot nylon net was treated with the aqueous composition. The net was allowed to soak for 5 minutes. The net was then removed and allowed to dry in air. Twenty-one grams of nylon net soaked up approximately 0.5 grams of the formulation.

### Field Testing:

Field tests were carried out in a low energy test site in the ocean bay. Control (triblock copolymer micellar solution) and treated (aqueous azadirachtin-encapsulated micellar solution) one square foot nylon nets were immersed at a depth of 18 feet. Net test samples were photographed after 20 weeks.

### Field Test Results:

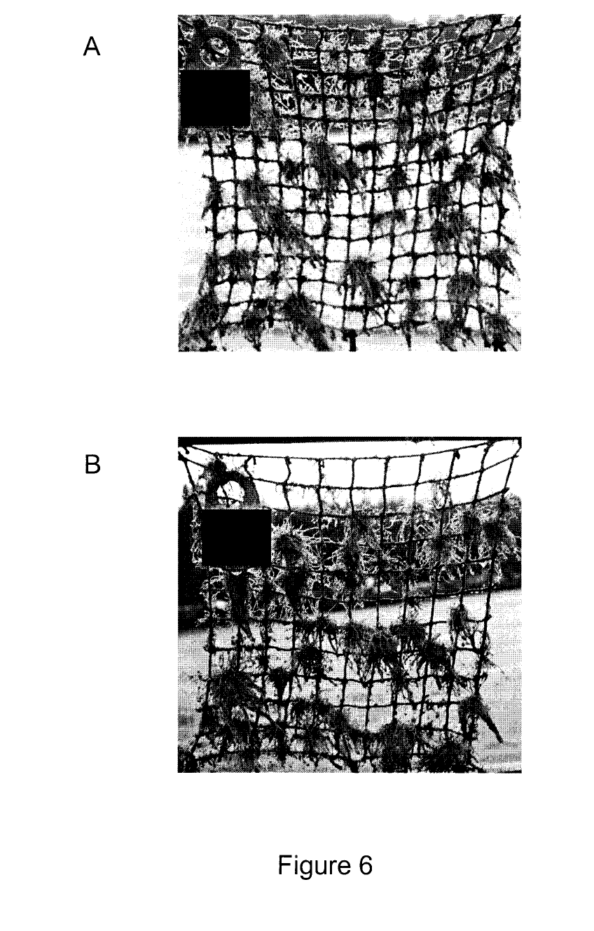

After 20 weeks in water, the control net was covered with hydroids (FIG. 6A), however, fouling was observed to be less than the untreated net (FIG. 5A). Macrofouling on the aqueous azadirachtin-encapsulated micellar solution treated net was reduced in comparison to the control (FIG. 6B).

Aqueous azadirachtin-encapsulated micellar solution treated nets showed antifouling properties, however, anti-macrofouling using this solution was not as great as that observed using neem oil alone. Without being bound to any theory, one possible reason for the observed difference in efficacy is that the triblock copolymer micelles are water-soluble and, hence, were not cross-linked to the net surface.

### Example 6

### Net Treatment:

Ten mL of neem oil (with increased azadirachtin concentration, 10,000 ppm) was mixed with 60 mL of polymerized linseed oil and 30 mL of limonene (end concentration of azadirachtin ˜1000 ppm). The mixture was stirred for half hour to allow thorough mixing. One square foot nylon net was treated with the mixed oil composition. The net was allowed to soak for 5 minutes. The net was then removed and allowed to air dry. Twenty-three grams of nylon net soaked up approximately 8 grams of the formulation.

### Field Testing:

Field tests were carried out in a low energy test site in the ocean bay. Combination neem oil/linseed oil/limonene treated (mixed formulation) one square foot nylon net was immersed at a depth of approximately 18 feet. Net test samples were photographed after 5 weeks.

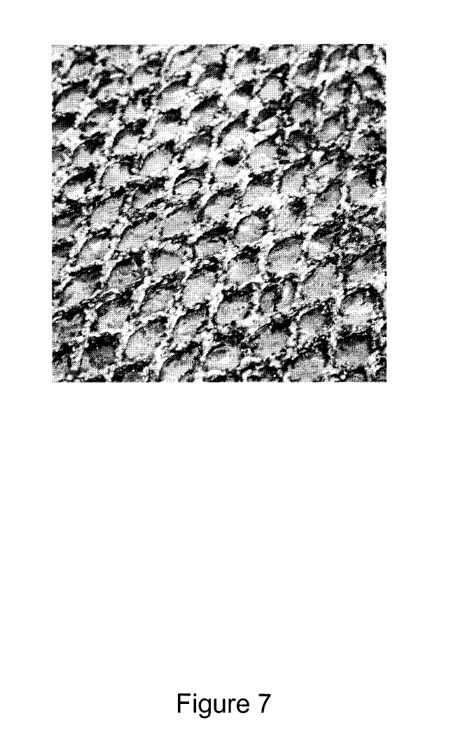

After 5 weeks in water, no observable macrofouling was detected on the treated net (FIG. 7). The mixed formulation treated nets showed very good efficacy against macrofouling species.

### Example 7 (Prophetic)

### Formulation: Foul Release Antifouling Treatment

Ten mL of neem oil (with increased azadirachtin concentration, 10,000 ppm) was mixed with 40 mL of 10% polyvinylpyrrolidone (for example, Kollidon® 30 or Kollidon® 90 (BASF)) prepared in N-methyl-2-pyrrolidone (NMP, Sigma Aldrich), 50 mL of aqueous polyurethane solution (for example, Bayhydrol® 2592 or Bayhydrol® 2593 (BASF), or Bondthane™ UD-250, 270, 302, or 610

(Bond Polymers International LLC)). The mixture was stirred for half hour to allow thorough mixing. One square foot nylon net was treated with this slippery antifouling composition. The net was allowed to soak for 5 minutes. The net was then removed and allowed to air dry. The net dry time was 4-5 hours, giving the net a smooth finish and greatly reducing the natural odor inherent to neem oil. When immersed in water, the net develops a slippery layer, providing a foul release mechanism along with antifouling properties.

### Example 8 (Prophetic)

### Formulation: Foul Release Antifouling Treatment

0.1 g of azadirachtin (1%) solid was mixed with 50 mL of 10% polyvinylpyrrolidone (for example, Kollidon® 30 or Kollidon® 90 (BASF)) prepared in N-methyl-2-pyrrolidone (NMP, Sigma Aldrich), 50 mL of aqueous polyurethane solution (for example, Bayhydrol® 2592 or Bayhydrol® 2593 (BASF), or Bondthane™ UD-250, 270, 302, or 610 (Bond Polymers International LLC)). The mixture was stirred for half hour to allow thorough mixing. One square foot nylon net was treated with this slippery antifouling composition. The net was allowed to soak for 5 minutes. The net was then removed and allowed to air dry. The net dry time was 4-5 hours, giving the net a smooth finish and greatly reducing the natural odor inherent to neem oil.

When immersed in water, the net develops a slippery layer, providing a foul release mechanism along with antifouling properties.

The foregoing description of the preferred embodiments of the invention has been presented to illustrate the principles of the invention and not to limit the invention to the particular embodiment illustrated. It is intended that the scope of the invention be defined by all of the embodiments encompassed within the claims. All documents referenced herein are incorporated by reference.

1. A composition comprising azadirachtin, preferably neem oil.

2. The composition of claim 1 further comprising at least one polymerized oil.

3. The composition of claim 2, wherein the at least one polymerized oil is selected from the group comprising a linseed oil, a perilla oil, a poppy seed oil, a soybean oil, a walnut oil, a tung oil, and mixtures thereof.

4. The composition of claim 1, wherein the composition comprises from 1 to 100% (v/v) neem oil.

5. The composition of claim 1, wherein the composition comprises azadirachtin in a concentration from at least 1 ppm to 65,000 ppm.

6. The composition of claim 2, wherein the composition comprises from >0 to 99% (v/v) of the at least one polymerized oil.

7. The composition of claim 2, wherein the at least one polymerized oil is a boiled oil having an iodine number greater than 120.

8. The composition of claim 1, further comprising at least one essential oil derived from a fruit or a flower.

9. The composition of claim 8, wherein the at least one essential oil is selected from the group comprising limonene, lavender, rose and mixtures thereof.

10. The composition of claim 1, further comprising linseed oil and limonene.

11. The composition of claim 1, further comprising a block-copolymer capable of forming a micelle.

12. The composition of claim 11, wherein the block-copolymer is a biodegradable block copolymer, preferably polycaprolactone-block-poly(ethylene oxide)-block polycaprolactone triblock copolymer.

13. The composition of claim 1, further comprising a foul release agent.

14. The composition of claim 13, wherein the foul release agent is polyvinylpyrrolidone.

15. A polymer soaked with the composition of claim 1.

16. The polymer of claim 15, being nylon in the form of a net.

17. A method of inhibiting macrofouling on at least one polymer in an aquatic environment comprising:
applying the composition of claim 1 to the polymer.

18. The method of claim 17, wherein the at least one polymer is selected from the group consisting of a synthetic or a natural polymer.

19. The method of claim 18, wherein the at least one polymer is selected from the group consisting of nylon, high density polyethylene (HDPE), polyester, polyurethane, Teflon, cellulose and polypropylene.

20. The method of claim 19, wherein the at least one polymer is contained in at least one of a fiber, a rope, a sheet, a film, and a net, preferably a net.

21. The method of claim 17, wherein the composition inhibits macrofouling of at least one aquatic plant or aquatic animal selected from the group comprising an hydroid, a mussel, a tunicate, a barnacle, a bryozoan, an annelid, and a macroalgae.

22. The method of claim 17, wherein the applying of the composition to the at least one polymer comprises at least one of dipping, spraying, brushing, rolling and pouring the composition over the at least one polymer.

23. The method of claim 17, wherein the composition is applied to the at least one polymer during the cold drawing stage of producing a polymer fiber.

24. The method of claim 23, wherein the cold drawing process is executed on a draw twister machine.

25-26. (canceled)
